SEQUENCE LISTING (97 sequences)

SEQ ID NO: 1 - protein, 22 aa, Homo sapiens
  MISC_FEATURE (1)..(22): Residues 50-70 of human heavy chain antibody germline sequence
RINPNSGGTNYAQKFQGRVTST

SEQ ID NO: 2 - protein, 22 aa, Homo sapiens
  MISC_FEATURE (1)..(22): Residues 50-70 of human heavy chain antibody germline sequence
WINAGNGNTKYSQKFQGRVTIT

SEQ ID NO: 3 - protein, 22 aa, Homo sapiens
  MISC_FEATURE (1)..(22): Residues 50-70 of human heavy chain antibody germline sequence
WMNPNSGNTGYAQKFQGRVTMT

SEQ ID NO: 4 - protein, 22 aa, Homo sapiens
  MISC_FEATURE (1)..(22): Residues 50-70 of human heavy chain antibody germline sequence
WISAYNGNTNYAQKLQGRVTMT

SEQ ID NO: 5 - protein, 22 aa, Homo sapiens
  MISC_FEATURE (1)..(22): Residues 50-70 of human heavy chain antibody germline sequence
GFDPEDGETIYAQKFQGRVTMT

SEQ ID NO: 6 - protein, 22 aa, Homo sapiens
  MISC_FEATURE (1)..(22): Residues 50-70 of human heavy chain antibody germline sequence
WITPFNGNTNYAQKFQDRVTIT

SEQ ID NO: 7 - protein, 22 aa, Homo sapiens
  MISC_FEATURE (1)..(22): Residues 50-70 of human heavy chain antibody germline sequence
IINPSGGSTSYAQKFQGRVTMT

SEQ ID NO: 8 - protein, 22 aa, Homo sapiens
  MISC_FEATURE (1)..(22): Residues 50-70 of human heavy chain antibody germline sequence
WIVVGSGNTNYAQKRQERVTIT

SEQ ID NO: 9 - protein, 22 aa, Homo sapiens
  MISC_FEATURE (1)..(22): Residues 50-70 of human heavy chain antibody germline sequence
GIIPIFGTANYAQKFQGRVTIT

SEQ ID NO: 10 - protein, 22 aa, Homo sapiens
  MISC_FEATURE (1)..(22): Residues 50-70 of human heavy chain antibody germline sequence
LVDPEDGETIYAEKFQGRVTIT

SEQ ID NO: 11 - protein, 21 aa, Homo sapiens
  MISC_FEATURE (1)..(21): Residues 50-70 of human heavy chain antibody germline sequence
LIYWNDDKRYSPSKLSRLTIT

SEQ ID NO: 12 - protein, 21 aa, Homo sapiens
  MISC_FEATURE (1)..(21): Residues 50-70 of human heavy chain antibody germline sequence
HIFSNDEKSYSTSLKSRLTIS

SEQ ID NO: 13 - protein, 21 aa, Homo sapiens
  MISC_FEATURE (1)..(21): Residues 50-70 of human heavy chain antibody germline sequence
LIDWDDDKYYSTSLKTRLTIS

SEQ ID NO: 14 - protein, 22 aa, Homo sapiens
  MISC_FEATURE (1)..(22): Residues 50-70 of human heavy chain antibody germline sequence
NIKQDGSEKYYVDSVKGRFTIS

SEQ ID NO: 15 - protein, 22 aa, Homo sapiens
GISWNSGSIGYADSVKGRFTIS

SEQ ID NO: 16 - protein, 22 aa, Homo sapiens
YISSSGSTIYYADSVKGRFTIS

SEQ ID NO: 17 - protein, 21 aa, Homo sapiens
  MISC_FEATURE (1)..(21): Residues 50-70 of human heavy chain antibody germline sequence
AIGTAGDTYYPGSVKGRFTIS

SEQ ID NO: 18 - protein, 24 aa, Homo sapiens
  MISC_FEATURE (1)..(24): Residues 50-70 of human heavy chain antibody germline sequence
RIKSKTDGGTTDYAAPVKGRFTIS

SEQ ID NO: 19 - protein, 22 aa, Homo sapiens
  MISC_FEATURE (1)..(22): Residues 50-70 of human heavy chain antibody germline sequence
GVSWNGSRTHYVDSVKRRFIIS

SEQ ID NO: 20 - protein, 22 aa, Homo sapiens
  MISC_FEATURE (1)..(22): Residues 50-70 of human heavy chain antibody germline sequence
GVSWNGSRTHYADSVKGRFIIS

SEQ ID NO: 21 - protein, 22 aa, Homo sapiens
GINWNGGSTGYADSVKGRFTIS

SEQ ID NO: 22 - protein, 22 aa, Homo sapiens
  MISC_FEATURE (1)..(22): Residues 50-70 of human heavy chain antibody germline sequence
SISSSSSYIYYADSVKGRFTIS

SEQ ID NO: 23 - protein, 22 aa, Homo sapiens
AISGSGGSTYYADSVKGRFTIS

SEQ ID NO: 24 - protein, 22 aa, Homo sapiens
  MISC_FEATURE (1)..(22): Residues 50-70 of human heavy chain antibody germline sequence
VISYDGSNKYYADSVKGRFTIS

SEQ ID NO: 25 - protein, 22 aa, Homo sapiens
  MISC_FEATURE (1)..(22): Residues 50-70 of human heavy chain antibody germline sequence
VISYDGSNKYYADSVKGRFTIS

SEQ ID NO: 26 - protein, 22 aa, Homo sapiens
  MISC_FEATURE (1)..(22): Residues 50-70 of human heavy chain antibody germline sequence
VIWYDGSNKYYADSVKGRFTIS

SEQ ID NO: 27 - protein, 22 aa, Homo sapiens
  MISC_FEATURE (1)..(22): Residues 50-70 of human heavy chain antibody germline sequence
GVSWNGSRTHYADSVKGRFIIS

SEQ ID NO: 28 - protein, 20 aa, Homo sapiens
  MISC_FEATURE (1)..(20): Residues 50-70 of human heavy chain antibody germline sequence
SISGGSTYYADSRKGRFTIS

SEQ ID NO: 29 - protein, 22 aa, Homo sapiens
  MISC_FEATURE (1)..(22): Residues 50-70 of human heavy chain antibody germline sequence
LISWDGGSTYYADSVKGRFTIS

SEQ ID NO: 30 - protein, 22 aa, Homo sapiens
  MISC_FEATURE (1)..(22): Residues 50-70 of human heavy chain antibody germline sequence
YISSSSSTIYYADSVKGRFTIS

SEQ ID NO: 31 - protein, 24 aa, Homo sapiens
  MISC_FEATURE (1)..(24): Residues 50-70 of human heavy chain antibody germline sequence
FIRSKAYGGTTEYTASVKGRFTIS

SEQ ID NO: 32 - protein, 21 aa, Homo sapiens
  MISC_FEATURE (1)..(21): Residues 50-70 of human heavy chain antibody germline sequence
VIYSGGSTYYADSVKGRFTIS

SEQ ID NO: 33 - protein, 22 aa, Homo sapiens
  MISC_FEATURE (1)..(22): Residues 50-70 of human heavy chain antibody germline sequence
AISSNGGSTYYANSVKGRFTIS

SEQ ID NO: 34 - protein, 21 aa, Homo sapiens
  MISC_FEATURE (1)..(22): Residues 50-70 of human heavy chain antibody germline sequence
VIYSGGSTYYADSVKGRFTIS

SEQ ID NO: 35 - protein, 24 aa, Homo sapiens
  MISC_FEATURE (1)..(24): Residues 50-70 of human heavy chain antibody germline sequence
RTRNKANSYTTEYAASVKGRFTIS

SEQ ID NO: 36 - protein, 24 aa, Homo sapiens
  MISC_FEATURE (1)..(24): Residues 50-70 of human heavy chain antibody germline sequence
RIRSKANSYATAYAASVKGRFTIS

SEQ ID NO: 37 - protein, 22 aa, Homo sapiens
  MISC_FEATURE (1)..(22): Residues 50-70 of human heavy chain antibody germline sequence
RINSDGSSTSYADSVKGRFTIS

SEQ ID NO: 38 - protein, 20 aa, Homo sapiens
  MISC_FEATURE (1)..(20): Residues 50-70 of human heavy chain antibody germline sequence
SISGGSTYYADSRKGRFTIS

SEQ ID NO: 39 - protein, 21 aa, Homo sapiens
  MISC_FEATURE (1)..(21): Residues 50-70 of human heavy chain antibody germline sequence
EIYHSGSTNYNPSLKSRVTIS

SEQ ID NO: 40 - protein, 21 aa, Homo sapiens
  MISC_FEATURE (1)..(22): Residues 50-70 of human heavy chain antibody germline sequence
YIYYSGSTYYNPSLKSRVTMS

SEQ ID NO: 41 - protein, 21 aa, Homo sapiens
  MISC_FEATURE (1)..(21): Residues 50-70 of human heavy chain antibody germline sequence
YIYHSGSTYYNPSLKSRVTIS

SEQ ID NO: 42 - protein, 21 aa, Homo sapiens
  MISC_FEATURE (1)..(21): Residues 50-70 of human heavy chain antibody germline sequence
YIYYSGSTYYNPSLKSRVTIS

SEQ ID NO: 43 - protein, 21 aa, Homo sapiens
  MISC_FEATURE (1)..(21): Residues 50-70 of human heavy chain antibody germline sequence
YIYYSGSTYYNPSLKSLVTIS

SEQ ID NO: 44 - protein, 21 aa, Homo sapiens
  MISC_FEATURE (1)..(21): Residues 50-70 of human heavy chain antibody germline sequence
EINHSGSTNYNPSLKSRVTIS

SEQ ID NO: 45 - protein, 21 aa, Homo sapiens
  MISC_FEATURE (1)..(21): Residues 50-70 of human heavy chain antibody germline sequence
SIYYSGSTYYNPSLKSRVTIS

SEQ ID NO: 46 - protein, 21 aa, Homo sapiens
  MISC_FEATURE (1)..(21): Residues 50-70 of human heavy chain antibody germline sequence
YIYYSGSTNYNPSLKSRVTIS

SEQ ID NO: 47 - protein, 21 aa, Homo sapiens
  MISC_FEATURE (1)..(21): Residues 50-70 of human heavy chain antibody germline sequence
YIYYSGSTNYNPSLKSRVTIS

SEQ ID NO: 48 - protein, 21 aa, Homo sapiens
  MISC_FEATURE (1)..(21): Residues 50-70 of human heavy chain antibody germline sequence
SIYHSGSTYYNPSLKSRVTIS

SEQ ID NO: 49 - protein, 22 aa, Homo sapiens
  MISC_FEATURE (1)..(22): Residues 50-70 of human heavy chain antibody germline sequence
IIYPGDSDTRYSPSFQGQVTIS

SEQ ID NO: 50 - protein, 22 aa, Homo sapiens
  MISC_FEATURE (1)..(22): Residues 50-70 of human heavy chain antibody germline sequence
RIDPSDSYTNYSPSFQGHVTIS

SEQ ID NO: 51 - protein, 23 aa, Homo sapiens
  MISC_FEATURE (1)..(23): Residues 50-70 of human heavy chain antibody germline sequence
RTYYRSKWYNDYAVSVKSRITIN

SEQ ID NO: 52 - protein, 22 aa, Homo sapiens
  MISC_FEATURE (1)..(22): Residues 50-70 of human heavy chain antibody germline sequence
WINTNTGNPTYAQGFTGRFVFS

SEQ ID NO: 53 - protein, 22 aa, Homo sapiens
  MISC_FEATURE (1)..(22): Residues 50-70 of human heavy chain antibody germline sequence
WFNTYTGNPTYAQGFTGRFVFS

SEQ ID NO: 54 - protein, 32 aa, Homo sapiens
  NON_CONS (1)..(32)
  MISC_FEATURE (1)..(32): Sequence template AA
VSHEYVANRPPVVENSTDVQEESVRLNVYVFV

SEQ ID NO: 55 - protein, 32 aa, Homo sapiens; identical to SEQ ID NO: 54
  MISC_FEATURE: Sequence 1DN2_A
  NON_CONS (1)..(32)

SEQ ID NO: 56 - protein, 32 aa, Homo sapiens
  MISC_FEATURE: Sequence AAL35303
  NON_CONS (1)..(32)
LDPEYVANRPPVVENGTDVQEESVRLNVSVIV

SEQ ID NO: 57 - protein, 32 aa, Homo sapiens
  MISC_FEATURE: Sequence CAC44624
  NON_CONS (1)..(32)
VSQEYVANKPPVVENSTDVQEDSVRLNVFVFV

SEQ ID NO: 58 - protein, 32 aa, Homo sapiens
  MISC_FEATURE: Sequence 1605217A
  NON_CONS (1)..(32)
VSHEYVADRPPVVENSTDVQEESVRLNVYVFV

SEQ ID NO: 59 - protein, 32 aa, Homo sapiens; identical to SEQ ID NO: 58
  MISC_FEATURE: Sequence AAG00448
  NON_CONS (1)..(32)

SEQ ID NO: 60 - protein, 32 aa, Homo sapiens; identical to SEQ ID NO: 58
  MISC_FEATURE: Sequence AAD38158
  NON_CONS (1)..(32)

SEQ ID NO: 61 - protein, 17 aa, Homo sapiens
TAVDRNHRGMRAWVLTT

SEQ ID NO: 62 - protein, 17 aa, Homo sapiens
AAVNRNHRGMRLWVLTT

SEQ ID NO: 63 - protein, 17 aa, Homo sapiens
AAVTRSHRGMWAWVLTT

SEQ ID NO: 64 - protein, 17 aa, Homo sapiens
AAVTRSHRGMWAWVLTT

SEQ ID NO: 65 - protein, 17 aa, Homo sapiens
AAVTRSHRGMIVWVLTT

SEQ ID NO: 66 - protein, 17 aa, Homo sapiens
AAVNRNHRGMALWVLTT

SEQ ID NO: 67 - protein, 17 aa, Homo sapiens
AAVNRNHRGMALWVLTS

SEQ ID NO: 68 - protein, 17 aa, Homo sapiens
AAVTRSSTGIWAWVLTT

SEQ ID NO: 69 - protein, 17 aa, Homo sapiens
AAVNRNHRGMVLWVLKT

SEQ ID NO: 70 - protein, 17 aa, Homo sapiens
AAVNRNHRGMVLWVLKT

SEQ ID NO: 71 - protein, 17 aa, Homo sapiens
AAVNRNHRGMVLWVLKT

SEQ ID NO: 72 - protein, 17 aa, Homo sapiens
AAVTRSNRGIWAWVLTT

SEQ ID NO: 73 - protein, 17 aa, Homo sapiens
AVVTTDHAGMLAWVLTT

SEQ ID NO: 74 - protein, 17 aa, Homo sapiens
AGVKRSSAGIRAWVLTT

SEQ ID NO: 75 - protein, 17 aa, Homo sapiens
AAVNKNHRGMLLWVLTS

SEQ ID NO: 76 - protein, 17 aa, Homo sapiens
AAMRRSHRGLWAWVLTT

SEQ ID NO: 77 - protein, 17 aa, Homo sapiens
AAVTRSNLGMWAWVLPT

SEQ ID NO: 78 - protein, 17 aa, Homo sapiens
AAVERSSAGIGAWVLAT

SEQ ID NO: 79 - protein, 17 aa, Homo sapiens
AAVTRSNMGMWAWVLPT

SEQ ID NO: 80 - protein, 17 aa, Homo sapiens
AAVNKNHRGMGLWVLIS

SEQ ID NO: 81 - protein, 17 aa, Homo sapiens
AAVDRNDRGMRLWVLTS

SEQ ID NO: 82 - protein, 17 aa, Homo sapiens
AAVNRNSRGMSLWILTT

SEQ ID NO: 83 - protein, 17 aa, Homo sapiens
AVVTTDHEGMGAWVLTT

SEQ ID NO: 84 - protein, 17 aa, Homo sapiens
AFVTRNSKGVRVWILKQ

SEQ ID NO: 85 - protein, 17 aa, Homo sapiens
AAVNRNSRGMVLWVLTT

SEQ ID NO: 86 - protein, 17 aa, Homo sapiens
AAVNRNSRGMVLWVLTT

SEQ ID NO: 87 - protein, 17 aa, Homo sapiens
AGVKRSGAGIIAWVLTT

SEQ ID NO: 88 - protein, 17 aa, Homo sapiens
VAVNRNNRGMGLWVLTT

SEQ ID NO: 89 - protein, 17 aa, Homo sapiens
AAVNRNNRGMSLWVLIS

SEQ ID NO: 90 - protein, 17 aa, Homo sapiens
AAVNRNSRGMGLWVLTS

SEQ ID NO: 91 - protein, 17 aa, Homo sapiens
AAVNRNNRGMYLWVLIS

SEQ ID NO: 92 - protein, 17 aa, Homo sapiens
AAVNKNSRGMALWVLTT

SEQ ID NO: 93 - protein, 17 aa, Homo sapiens
TAVDTNSRGMRLWVLTT

SEQ ID NO: 94 - protein, 17 aa, Homo sapiens
AAVNRNSRGMNLWVLKS

SEQ ID NO: 95 - protein, 17 aa, Homo sapiens
KAVNKNSRGMSLWVLTT

SEQ ID NO: 96 - protein, 17 aa, Homo sapiens
AVVTRNSVGWYFWILTQ

SEQ ID NO: 97 - protein, 17 aa, Mus musculus
SAVTRNHAGIRAWVLTT

(19) United States
(12) Patent Application Publication
(10) Pub. No.: US 2007/0249809 A1
(43) Pub. Date: Oct. 25, 2007

(54) Protein engineering with analogous contact environments

(75) Inventor: John R. Desjarlais, Pasadena, CA (US)

Correspondence Address: MORGAN, LEWIS & BOCKIUS, LLP, ONE MARKET SPEAR STREET TOWER, SAN FRANCISCO, CA, 94105, US

(73) Assignee: Xencor, Inc.

(21) Appl. No.: 11/488,984

(22) Filed: Jul. 18, 2006

Related U.S. Application Data
(63) Continuation of application No. 11/008,647, filed on Dec. 8, 2004.
(60) Provisional application No. 60/528,230, filed on Dec. 8, 2003.
(60) Provisional application No. 60/602,566, filed on Aug. 17, 2004.

(51) Int. Cl. C07K 1/00

(52) U.S. Cl. 530/350

(57) ABSTRACT

The invention relates to novel methods for engineering protein sequences using structural and homology information.

This application is a continuation application of U.S. application Ser. No. 11/008,647, filed Dec. 8, 2004, which claims of benefit under 35 U.S.C. §119(e) to USSNs 60/528,230, filed Dec. 8, 2003 and 60/602,566, filed Aug. 17, 2004, all which are hereby incorporated by reference in their entireties.

### FIELD OF THE INVENTION

The invention relates to novel methods for engineering protein sequences using structural and homology information.

### BACKGROUND OF THE INVENTION

Throughout evolution, the processes of genetic drift and natural selection have lead to the exploration of countless protein sequences, many with related structures and functions. Using well-known methods of bioinformatics, most naturally occurring protein sequences may be aligned relative to homologues that have related sequences and structures. Ultimately, one creates a multiple sequence alignment (MSA) of numerous members of a protein family, using any of a variety of sequence or structure alignment programs known in the art. A great deal of useful information exists in these sets of related proteins and their sequences. Because they have similar structures and functions, an amino acid found at a particular position in one member of a protein family may be a useful substitution at an equivalent position in an alternative member of the family. Modification of the amino acid sequence of a protein is frequently used to create variant proteins with improved properties, including proteins with higher stability, altered specificity, and altered activity. However, such a strategy often fails due to the complex nature of protein structure and evolutionary sequence changes. An amino acid that is favorable in one protein can thus be unfavorable in a related protein. This issue most typically arises because of strong coupling patterns between two or more amino acids that closely interact in the three-dimensional structure of the protein. Hence, there is a need in the art to more optimally utilize information from multiple sequence alignments.

Accordingly, it is an object of the invention to provide methods for analysis and comparison of related proteins to predict the compatibility or feasibility of novel amino acid sequences with a specified protein structural form. It is an object of the invention to provide methods for combining sequence alignment information with structural information in order to evaluate the compatibility of amino acid combinations within a given protein structural form. It is an object of the present invention to further provide sequence and structure-based scoring functions that may be used to evaluate the fitness of substitutions in a template protein. In a preferred embodiment, said scoring functions evaluate one or more substitutions for their structural compatibility with a protein structure template. It is a further object of the invention to predict structural compatibility by combining sequence alignment information with structural information. The invention finds use in various contexts in which prediction of favorable protein sequences is desired, for example protein engineering including antibody engineering, humanization of antibodies, CDR grafting, chimeric protein creation, the transfer of active site or binding sites, protein stability or specificity prediction, protein identification from databases, or various other protein design and bioinformatics projects. The methods described herein are part of the ACE™ methods, or Analogous Contact Environment methods.

### SUMMARY OF THE INVENTION

Thus, the present invention provides methods for modifying a first protein to generate a second protein, comprising comparing a structural environment of at least one reference position of the first protein and at least one structural environment of the corresponding at least one reference position of at least one related protein. In some aspects, a number of related proteins are used or tested, with from 5 to 10 to 50 to 100 different related proteins all being preferred. A scoring function is then used to generate a score for the similarity of said structural environment of said at least one related protein to said structural environment of said first protein. At least one modification for said at least one reference position of said first protein to generate said second protein is selected. The scoring function comprises use of a proximity measure. In some aspects, the structural environments can include single positions (e.g. amino acids) or a plurality of positions.

The scoring function can include a number of components, including the use of proximity values of directly contacting amino acids and indirectly contacting amino acids, evaluation of amino acid similarity values, a simultaneous comparison of proximity values and amino acid similarity values, a non-discrete proximity function, a non-binary comparison of environment similarity, a non-binary comparison of amino acid similarities, structural precedence scores, and relative environmental similarity scores.

In an additional aspect, the method utilizes a frequency function wherein the frequency function uses multiple scores from said scoring function.

In a further aspect, the amino acid chosen to be modified is chosen based on at least two measures selected from the following: structure-weighted frequency, relative environmental similarity, and precedence.

In an additional aspect, modifications are chosen based on the highest similarity score, or on a score in the highest 10 to 50%.

In a further aspect, the invention provides methods for modifying a first protein to generate a second protein, comprising:
(a) comparing a structural environment of at least two reference positions of said first protein and at least one structural environment of the corresponding at least two reference positions of at least one related protein;
(b) using a scoring function to generate a score for the similarity of said structural environment of said at least one related protein to said structural environment of said first protein; and,
(c) selecting at least two modifications for said at least two reference positions of said first protein to generate said second protein;
(d) wherein said scoring function comprises use of a proximity measure.
(e) In a further aspect, the invention provides methods for modifying a first protein to generate a second protein, comprising:
(f) comparing a structural environment of at least two reference positions of said first protein and at least one structural environment of the corresponding reference positions of at least two related proteins;
(g) using a scoring function to generate a score for the similarity of said structural environment of said at least one related protein to said structural environment of said first protein;
(h) selecting one related protein comprising a similar structural environment to said first protein; and,
(i) selecting at least two modifications for said at least two reference positions of said first protein to generate said second protein;
(j) wherein said scoring function comprises use of a proximity measure.

In an additional aspect, the invention provides methods for modifying a first protein to generate a second protein, comprising:
(a) comparing a structural environment of at least two reference positions of a template protein and at least one structural environment of the corresponding reference positions of at least one related protein;
(b) using a scoring function to generate a score for the similarity of said structural environment of said template protein to said structural environment of said related protein;
(c) selecting said first protein comprising a similar structural environment to said template protein from said related proteins; and,
(d) selecting at least two modifications for said at least two reference positions of said first protein to generate said second protein;
(e) wherein said scoring function comprises use of a proximity measure.

In a further aspect, the invention provides methods for modifying a first protein to generate a second protein, comprising:
(a) comparing a structural environment of at least one reference position of said first protein and at least one structural environment of the corresponding at least one reference position of at least one related protein;
(b) selecting at least one modification for said reference position of said first protein to generate said second protein.

In an additional aspect, the invention provides methods for modifying a first protein to generate a second protein, comprising:
(a) comparing a structural environment of at least one reference position of said first protein and at least one structural environment of the corresponding at least one reference position of at least one related protein;
(b) using a scoring function to generate a score for the similarity of said structural environment of said related protein to said structural environment of said first protein; and,
(c) selecting at least one modification for said reference position of said first protein to generate said second protein.

In a further aspect, the invention provides of generating a variant protein sequence comprising:
(a) inputting a structure comprising at least a first structural environment of a first set of reference amino acid positions of a first protein into a computer;
(b) identifying the corresponding second structural environment of a second set of reference amino acid positions of said second protein;
(c) using a computational scoring function comprising a proximity measure to generate a score for the similarity of said first and second structural environments;
(d) using said score to identify variant amino acid residues to replace at least one amino acid at one of said positions in said first set;
(e) generating at least one variant protein sequence comprising at least one of said variant amino acid residues to generate a variant protein.

In an additional aspect, the invention provides methods as above further comprising providing a sequence of a third related protein and using said scoring function to generate a score for the similarity of a third structural environment of a third set of reference amino acid positions of said third protein to said first structural environment. That is, structural environments of two related proteins are compared to the first protein. The method may further comprise identifying the structural environment that is similar to said first structural environment, wherein said variant protein sequence comprises at least two of said variant amino acid residues.

In a further aspect, the method can further comprise using said scoring function to generate a score for the similarity of a third structural environment of a third set of reference amino acid positions of said first protein to a fourth structural environment of a corresponding fourth set of reference amino acid positions of said second protein, and using said score is used to identify variant amino acid residues to replace at least one amino acid at one of said positions in said first set and to replace at least one amino acid at one of said positions in said third set.

As for all the aspects outlined herein, the sets may independently contain one amino acid position or a plurality, in either linear sequence form or steric relatedness. In addition, one or more of the protein sequences (e.g. the first protein sequence or one or more of the related sequences) is a consensus sequence, a wild-type sequence, or a variant sequence.

### BRIEF DESCRIPTION OF THE DRAWINGS

FIG. 1. A portion of a multiple sequence alignment of human heavy chain antibody germline sequences (numbering is according to the Kabat system). Residues 50 to 70 are shown for 57 different sequences (SEQ ID NO:1-53).

FIG. 2. A schematic of an embodiment of the present invention. When assessing the potential for various amino acids to fit at a reference position (X), the template sequence and structure are compared to homologous proteins in the same family (A and B). The comparison is performed such that amino acids most structurally proximal to the reference position are most important. Thus, although homologue B has a more similar sequence overall (4 out of 6 identities with template), homologue A has a more similar sequence near the reference position, suggesting that F is a superior substitution to V at position X.

FIG. 3. Structure-weighted frequencies, or probabilities, for amino acid substitutions in m4D5 for reference positions 50 through 70. The upper matrix was calculated using the method of the present invention. The lower matrix was calculated using an unweighted frequency count of amino acids observed at each position in the alignment. An underscore in the top row indicates that the reference sequence, m4D5, contains a gap in that position of the multiple sequence alignment. For most positions, the probabilities generated using either method are substantially different. For example, at position 63, the method of the invention predicts that F and L are favorable amino acids, and that V is less favorable. In contrast, the simple, unweighted, counting method predicts that V is the most favorable.

FIG. 4. Illustration of an embodiment of the invention. A patch of residues may be defined. Proteins in the MSA may be screened to identify one that presents a similar patch environment to the patch environment of the original protein. In this example, the environment of protein 5 best matches the environment of the reference protein. Two possible implementations of the results are also shown. First, the patch of the reference protein may be transferred into the environment of protein 5 to create a new protein. Second, the patch of protein 5 may be transferred into the environment of the reference protein.

FIG. 5. Proximity values for two reference positions (129 and F68) in the structure of Herceptin® (trastuzumab) (Genentech/Biogenldec) (pdb accession code 1 FVC). 129 and F68 are shown as non-spherical surfaces. Calculated proximity values for all positions in the protein are mapped onto the structure by placing spheres on the Cα coordinate of each position. The volume of each sphere is proportional to the calculated proximity value (calculated with σ=5).

FIG. 6. Sequence weights for each human antibody heavy chain germline sequence at reference positions 50 through 70 (Kabat numbering), according to the template sequence m4D5 (heavy chain). The σ value of 5 was used in the proximity calculation (Eq. 1), the similarity matrix was BLOSUM62 (Eq. 2), and the temperature factor T was 1 (Eq. 3). Note that the sequence with the highest weight is strongly dependent on the reference position. For instance, although the germline sequence vh_{—}1-45 has the most similar environment to m4D5 at position 50, vh_{—}1-f has the most similar environment to m4D5 at position 51. Shading is used to highlight the larger sequence weights.

FIG. 7. Resim scores found using an embodiment of the present invention that determines the suitability of an environment for a patch of residues. In this example, the aligned sequences are antibody Fc domains and the representative structure was the Fc domain of human IgG1 (PDB code 1DN2) (SEQ ID NOS:54-60). The patch residues were in positions 266, 267, 268, 269 and 300. The five sequences from the MSA with the best environments for this patch are shown (SEQ ID NOS:56-60). The best environment comes from sequence, AAL35303 (SEQ ID NO:56), which differs from the template, 1 DN2 (SEQ ID NO:55), sequence at positions 298, 296, and 275.

FIG. 8. A comparison of CDR grafting using the methods of the present invention and a traditional method. The table shows patch resim scores of the present invention for graphing murine heavy chain CDR's onto various human heavy chain germine sequences. Resim scores of the present invention are shown in the first column for the top 28 scoring human sequences. The third and fourth columns show the percent identity of the human germline sequences to the murine sequence, a commonly used measure used to determine the best human sequence to accept the CDR graft. The percent identity is shown calculated in two manners. In column 3, the percent identity was calculated using all the residues in the variable heavy domain, whereas in column 4 the calculation used only the non-CDR residues in the variable heavy domain. The percent identity values suggest that h_vh_{—}1-2 and h_vh_{—}1-3 are the best acceptor sequences for this CDR graft.

FIG. 9. A graph showing that the methods of the present invention provide distinct information from previous methods of determining the appropriate acceptor sequence for a CDR graph. Plotted are the resim scores and the percent identities calculated for 52 variable heavy chain germline sequences. The resim scores of the present invention do not correlate well with percent identities, demonstrating that the methods of the present invention provide novel information useful in determining the best human acceptor sequence to receive the CDR graft.

FIG. 10. A table showing heavy chain germline amino acids found in the positions most proximal to the CDR regions. The reference sequence, the murine sequence (SEQ ID NO:97), for the CDR graft is shown in the top row. Amino acids in the human germline sequences (SEQ ID NOS:61-96) that differ from the donor, murine amino acids are shown in bold type. The proximities of each position to the CDR graft region, or patch, are shown in decreasing order toward the right. Resim scores for each possible acceptor sequence are shown in the first column.

### DESCRIPTION OF THE INVENTION

The present invention finds utility in identifying exchangeable or importable portions (including single amino acids) of related proteins based on the use of a scoring function, generally a multiparameteric scoring function, to score the similarity of the structural environments of reference positions with the starting protein and one or more related proteins. As is described herein, the present invention can be used in the design of variant proteins, which contain at least one modification as compared to a pre-existing protein. “Modification” in this sense means the insertion, deletion or substitution of any atoms or collections of atoms, most particularly amino acids. That is, in preferred embodiments, the modification is the insertion, deletion or substitution of amino acids.

In the present invention, a particular position or region of the protein is designated as the “reference position”. In the case of multiple amino acids, for example, this is sometimes referred to a “patch”. The reference or patch region can contain one or more positions in the protein. One aspect of the present invention is the assessment of the compatibility of a reference region of a first protein and one or more structural environment regions of a second, related protein. That is, by using the scoring function as defined herein, the similarity of the structural environment of a first region of a first protein is compared to the structural environment of the corresponding region in a second related protein. Depending on the desired objective, the reference, or patch, region may be considered the “variable” region or the “fixed” region in a protein design. Likewise, the environmental positions may be considered either “variable” or “fixed” depending on the application of the present invention.

For example, one application of the present invention is in CDR grafting. In this design procedure, the CDR (complement-determining region) sequences from one antibody, for example a murine antibody, are substituted into another antibody, for example, a human antibody. With this procedure, a novel antibody molecule can be formed that retains the antigen specificity of the murine antibody yet has reduced immunogenicity as compared to the murine antibody. In this example, the murine CDR regions may be considered fixed and can be designated as the patch of residues in the present invention. The algorithms in the present invention are used to determine the human antibody with the best environment in which to place the patch residues. In this case, the human environment residues would be considered variable. An alternative view of the same procedure is that the human antibody will have its CDR sequences replaced with those of the murine antibody. In this case, the CDR sequences may be considered variable and the remaining positions are considered fixed. Viewed in this manner, the patch residues, the CDR residues, are considered variable. In short, the technology of the present invention may be used to judge the compatibility of the patch residues and the remaining environment residues. For a given protein design goal, the patch residues could be considered fixed and the environment residues variable or the patch residues could be considered variable and the environment residues fixed.

Thus, by comparing structural environments of reference position(s) within a first protein with the corresponding reference position(s) of one or more related proteins, suitably similar structural environments are identified by using a scoring function to generate a similarity score. Once a suitable similar environment is identified by a suitable score, putative variable amino acid positions and/or variant residues at those positions are identified to replace corresponding residues in the first protein. One or more variant protein sequences (either as sequences or as physical proteins) can then be generated. These variants thus contain a modified structural environment at the reference position, as components of the environment have been modified to conform with the corresponding structural environment of the second (related) protein.

In addition, this process may be done using a first protein and a set of related proteins, or a single related protein. In the case of sets of related proteins, it may not be necessary to utilize additional structural information; for example, utilizing the structural information for the first protein, and using sequence alignment techniques to graft additional sequences onto the structure can be done. Similarly, this process may be done either simultaneously or sequentially on two reference positions or “patches” within the first protein and the related protein(s).

In addition, while the discussion below generally relates to the use of amino acids in the analysis, it should be recognized that other structural environments of a reference point, including but not limited to additional components of a structural environment of a protein such as the PEGylation structures, fatty acid structures, or glycosylation structures, can be used as to define the structural environments of interest.

In order that the invention may be more completely understood, several definitions are set forth below. Such definitions are meant to encompass grammatical equivalents.

By “protein” herein is meant at least two amino acids linked together by a peptide bond. As used herein, protein includes proteins, oligopeptides and peptides, and includes wild-type proteins, variant proteins, and fragments of either. The peptidyl group may comprise naturally occurring amino acids and peptide bonds, or synthetic peptidomimetic structures, i.e. “analogs”, such as peptoids (see Simon et al., PNAS USA 89(20):9367 (1992)). The amino acids may either be naturally occurring or non-naturally occurring; as will be appreciated by those in the art, any structure for which a set of rotamers is known or can be generated can be used as an amino acid. The side chains may be in either the (R) or the (S) configuration. In a preferred embodiment, the amino acids are in the (S) or L-configuration. The protein may be any protein for which a three dimensional structure is known or can be generated; that is, for which there are three-dimensional coordinates for each atom of the protein. The structure of the protein is not necessary for using the protein in the present invention. Generally structures can be determined using X-ray crystallographic techniques, NMR techniques, de novo modeling, homology modeling, etc. In general, if X-ray structures are used, structures at 2 Angstrom resolution or better are preferred, but not required. The proteins may be from any organism, including prokaryotes and eukaryotes, with enzymes from bacteria, fungi, extremeophiles such as the archebacteria, insects, fish, animals (particularly mammals and particularly human) and birds all possible.

Suitable proteins (both “starting” or “first” proteins and related protein(s)) include, but are not limited to, industrial, agricultural and pharmaceutical proteins, including ligands, cell surface receptors, antigens, antibodies, cytokines, hormones, and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database. Suitable protein backbones include, but are not limited to, all of those found in the protein database compiled and serviced by the Brookhaven National Lab. Specifically included within “protein” are fragments and domains of known proteins, including functional domains such as enzymatic domains, binding domains, etc., and smaller fragments, such as turns, loops, etc. That is, portions of proteins may be used as well.

In some embodiments, the starting proteins and/or related proteins are naturally occurring, e.g. wild-type proteins. Alternatively, the starting protein may be a consensus sequence of a family, and the related proteins are either members of the family or variants thereof.

By “structural environment” herein is meant a region of atoms surrounding one or more specified reference positions of a protein. The structural environment is preferably defined with higher emphasis for atoms that are closer in space to the reference position and lower emphasis for atoms that are farther in space from the reference position. In a more preferred embodiment, the atoms are components of amino acids. In a preferred embodiment, the structural environment constitutes atoms within 0 to 30 Angstroms from the reference position(s), with atoms with 0 to 15 generally being preferred. In some cases, the entire protein may be considered the structural environment of a particular residue or patch.

By “structural information” herein is meant three-dimensional information derived from at least one protein structure. In a preferred embodiment, structural information can atomic coordinates as derived using x-ray crystallographic methods, NMR methods, or the like. In additional embodiments, structural information can be in the form of interatomic distances; inter-side chain distances; Cα-Cα distances; or Cβ-Cβ distances; proximity values; a contact matrix; or consensus information from at least two related protein structures or domains can be used.

The protein backbone structure that is used can either include the coordinates for both the backbone and the amino acid side chains, or just the backbone, i.e. with the coordinates for the amino acid side chains removed. If the former is done, the side chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

The protein backbone structure contains at least one variable residue position. As is known in the art, the residues, or amino acids, of proteins are generally sequentially numbered starting with the N-terminus of the protein. Thus a protein having a methionine at its N-terminus is said to have a methionine at residue or amino acid position 1, with the next residues as 2, 3, 4, etc. At each position, the wild type (i.e. naturally occurring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer. These variable residue positions are generally identified herein as being part of the structural environment of interest.

In a preferred embodiment, all of the residue positions of the protein are variable. That is, every amino acid side chain may be altered in the methods of the present invention. This is particularly desirable for smaller proteins, although the present methods allow the design of larger proteins as well.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid. Alternatively, the methods of the present invention may be used to evaluate mutations de novo, as is discussed below.

In a preferred embodiment, residues which can be fixed include, but are not limited to, structurally or biologically functional residues. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, met al binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

As will be appreciated by those in the art, the methods of the present invention allow computational testing of “site-directed mutagenesis” targets without actually making the mutants, or prior to making the mutants. That is, quick analysis of sequences in which a small number of residues are changed can be done to evaluate whether a proposed change is desirable. In addition, this may be done on a known protein, or on a protein optimized as described herein.

As will be appreciated by those in the art, a domain of a larger protein may essentially be treated as a small independent protein; that is, a structural or functional domain of a large protein may have minimal interactions with the remainder of the protein and may essentially be treated as if it were autonomous. In this embodiment, all or part of the residues of the domain may be variable.

It should be noted that even if a position is chosen as a variable position, it is possible that the methods of the invention will optimize the sequence in such a way as to select the wild type residue at the variable position. This generally occurs more frequently for core residues, and less regularly for surface residues. In addition, it is possible to fix residues as non-wild type amino acids as well.

A “multiple sequence alignment (MSA)” is a collection of linear sequences in which a correspondence is established between the positions in the sequences. Each sequence in the MSA consists of a linear array of any type of element, with amino acids and nucleic acids being commonly used elements. The correspondence between elements in different sequences is commonly established by their relationship in the MSA. Alternatively, in the case of protein sequences, the correspondence can be established based on the 3-dimensional position of the amino acids in the protein structures, a “structure-based alignment”. MSA can come from a variety of sources, including databases and their generation from computer algorithms. Examples include, BLAST, PSI-BLAST (National Center for Biotechnology Information, National Institute of Health. U.S.A., Altschul, S. F. et al. (1990) J. Mol. Biol. 215:403-410) and CE (Shindyalov and Bourne (1998) Protein Engineering 11(9) 739-747). SCOP (Murzin A. G. et al. (1995). J. Mol. Biol. 247, 536-540), CATH (Orengo, C. A. (1997) Structure. 5(8):1093-1108), PFAM (Bateman, A et al. Nucleic Acids Research (2004) Issue 32:D138-D141), CLUSTALW (Chenna et al., Nucleic Acids Res. 31(13):3497-3500 (2003)), and BLOCKS (Henikoff et al. Nucleic Acids Res. 28:228-230 (2000)).

A “template” as used herein is simply a structure or sequence that is used as a reference to be compared to another structure or sequence.

A “similarity matrix” is a matrix of values establishing the degree of similarity of various elements. The elements may be, for example, the 20 commonly found amino acids, all the natural and unnatural amino acids, other molecules such as sugars and fatty acids, or other entities. In the case wherein the similarity matrix is used to compare amino acids, a value in a certain row and column describes the similarity between the amino acid representing that row and the amino acid representing that column. The values in a similarity matrix can be derived from essentially any property of the elements found in the rows and columns. Properties of amino acids used include substitution frequencies in protein families, hydrophobicity, size and charge. Similarity matrices based on amino acid substitution frequencies are the most preferred in the present invention and include BLOSUM and PAM matrices (Henikoff S and Henikoff H. G. Proc Natl Acad Sci USA. 1992 Nov. 15; 89(22):10915-9; Dayhoff M. R. et al. (1978) Atlas of Protein Sequences and Structure 5:345-352). Variations of similarity matrices that are specific for a particular protein family or class, e.g. membrane proteins, may also be used in the present invention.

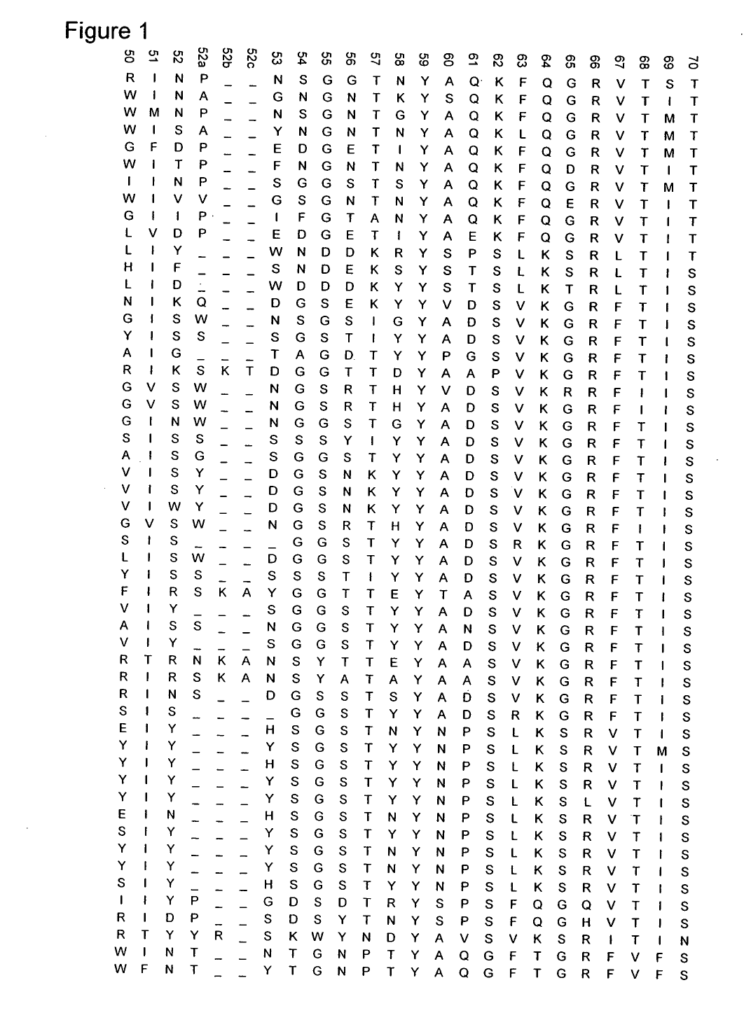

As is known in the art, multiple sequence alignments contain a wealth of information about a set of proteins. Proteins with similar sequences can be aligned to establish which residue in one protein corresponds to another residue in a related protein. Proteins that are similar in sequence often share a common structure or common function and therefore, multiple sequence alignments allow structurally or functionally important residues in a protein to be identified based on knowledge of a related protein. In protein design, the amino acid that could be substituted for another at a particular position in a protein may be decided by using an amino acid found in the corresponding position in a similar protein. If an amino acid has a high frequency at a position in a multiple sequence alignment, that amino acid is said to be “conserved” and the residue is likely to be important for the structure or function of the protein. FIG. 1 shows a multiple sequence alignment of human heavy chain antibody germline sequences. A strength of the present invention is its combining of information from multiple sequence alignments and protein structures to assess the fitness of an amino acid, or a set of amino acids, for a particular location in a protein.

A feature of a preferred embodiment is a description of an environment surrounding the amino acid(s) in question (the structural environment), and the use of environment comparisons within related proteins to provide quantitative predictions regarding the compatibility of specific amino acid combinations with the structure in question. The environment comprises many amino acids, each of which contributes to the environment according to its individual properties. In creating the environment, the properties considered by the algorithm comprise the similarity of substituting amino acids, the proximity of the environmental residues to the reference position(s) in question, and the overall similarity of the sequences.

A typical output of a preferred embodiment is a set of amino acid compatibility or precedence scores for at least one reference position of at least one protein. Extension of this to all reference positions of a protein leads to the definition of a matrix of probabilities and precedence scores denoting the structural compatibility of each amino acid type within each position of a template protein sequence. In an additional embodiment of the present invention, the compatibility of a set of amino acids, a “patch”, and the template protein is assessed. Structural compatibility probabilities for a given position are obtained by taking a weighted frequency count of amino acids observed at equivalent positions in a multiple sequence alignment of related proteins. Structural precedence values are obtained by assessing whether a similar arrangement of amino acids has been observed in an existing protein sequence. The weighting functions are derived by integrating information from the template sequence, each sequence in the MSA, and the three-dimensional structure(s) of one or more members of the protein family.

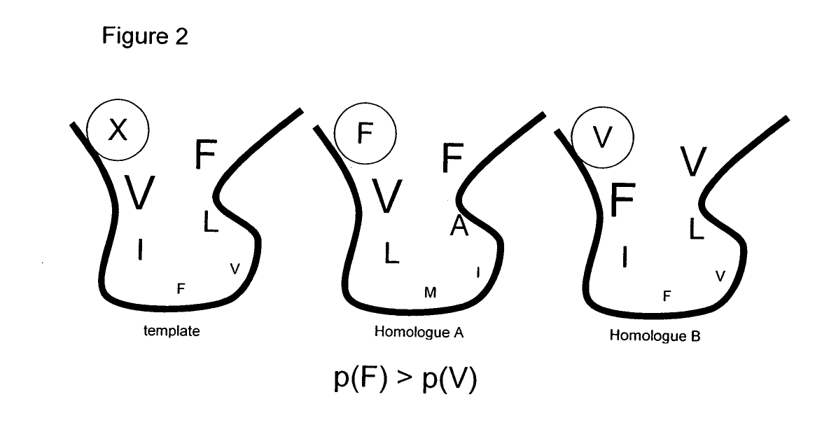

A more typical approach to utilizing MSA information is to take an unweighted frequency count of amino acids observed at equivalent positions in a MSA of related proteins. As is known in the art, this approach can be modified slightly by weighting the contribution of each MSA sequence to the statistics according to its overall dissimilarity to other sequences in the alignment (e.g. as in Henikoff and Henikoff, J Mol Biol. 1994 Nov. 4; 243(4):574-8). Unfortunately, this type of analysis is incomplete, leading in many cases to inaccurate predictions. The present invention adds two important features to this type of analysis. First, the similarity of the template sequence to each sequence in the MSA is considered and contributes to the weighted frequency count. Second, and most importantly, three-dimensional structure information contributes to the weighting procedure: similarities between the template sequence and each MSA sequence are assessed with increased influence for positions that are structurally proximal to the reference position. Thus, if protein A, related to the template protein, has a similar structural environment in the vicinity of reference position X, then the best choice of substitution at position X is the amino found at the corresponding reference position in protein A (FIG. 2).

In preferred embodiments, analysis comprises the steps of: (a) generating or obtaining a sequence alignment between a first protein and at least one related protein; (b) comparing a first protein and at least one related protein in the structural environment of at least one reference position; (c) evaluating similarity of structural environments between the first protein and at least one related protein (d) using environment similarity scores of each aligned related protein to quantify favorability or compatibility of amino acids at each reference position. It should be emphasized that equivalence or correspondence of reference positions is defined simultaneously for the first protein and each related protein according to the sequence alignment. The structural environment is established using positional proximity measures to the reference position(s), generally applied such that the structural environment predominantly constitutes positions close in space to the reference position, while de-emphasizing or excluding positions farther in space from the reference position. Favorability or compatibility information for various amino acids at the reference positions can ultimately be used to select judicious substitutions, predict the stability of various sequences, or to predict interaction affinities (e.g. if the analysis is extended to include a multi-subunit proteins or protein-protein and protein-peptide complexes).

In preferred embodiments, analysis includes the use of a multiple sequence alignment (MSA) comprising the first protein and several related proteins, generating reference position weights for each sequence in the MSA by scoring similarities between the reference position environment of the first protein and corresponding reference position environments of each MSA sequence, and generating probability or structural precedence values for each amino acid at each reference position. In general, more MSA sequences are desirable for the most accurate predictions. However, in some circumstances, small numbers of related proteins can be used.

A variety of methods can be applied to evaluate the similarity of two structural environments (one from a first protein and one from a related protein) surrounding equivalent reference positions. The evaluation will generally involve an analysis of the amino acid content of the structural environment and the spatial distribution of amino acids around the reference position (in some embodiments, other chemical entities may be included), and in some cases will further involve analysis of the atomic content and spatial distribution of atoms around the reference position. For example, for cases in which three-dimensional atomistic structures are known or can be constructed for the sequences in a given MSA, atomistic coordinates can be used to calculate environment similarity. In an alternative embodiment, environment similarity may be calculated by comparing the three-dimensional coordinates of the atoms of the amino acids. The comparison may include the root-mean-squared distance (RMSD) between the coordinates of the amino acid sidechains, the difference in amino acid side-chain dihedral angle values, the amount of overlapping occupied volume shared between the amino acid side-chains, the extent of coordinate overlap of atoms with similar physico-chemical properties (e.g. charge, polarizability, size, and hydrogen-bonding capacity) or the like.

In preferred embodiments, similarity of structural environment may be evaluated and scored using proximity values—between each environment amino acid position and the reference position—and combined with amino acid similarity comparisons for amino acids in the structural environment.

In a preferred embodiment, proximities are derived from a position-position distances calculated from three-dimensional structures of one or more members of the protein family. Methods for calculating a matrix of side-chain side-chain or position-position distances from a protein structure are well known in the art. These include but are not limited to C_{α}-C_{α} and C_{β}-C_{α} distance matrices. In preferred embodiments, centroid-centroid distances are calculated. In alternative preferred embodiments, the average of all side chain-side chain interatomic distances are calculated to yield a contact distance between each pair of side chains in a protein structure. In alternative embodiments, distances are calculated as the point of closest approach of the two side chain atoms. In additional embodiments, one or more distance matrices can be averaged (after appropriate alignment of the matrices to account for gaps/insertions in the different protein structures).

In a preferred embodiment, distance values are converted to structural proximity measures or values.

In a preferred embodiment, structural proximity is calculated with a function that decreases as a function of distance. Examples include, but are not limited to, Gaussian functions (as in Eq. 1), decreasing sigmoidal functions, exponential decay functions, and step functions.

$$Equation 1 \text{:}$$

$${proximity}_{ij} = {ⅇ}^{- {( \frac{{d}_{ij}}{σ} )}^{2}}$$

In preferred embodiments a Gaussian σ value between 4 and 10 is preferred, with 5 being especially preferred, although other values may be optimal in some situations. This and other embodiments place highest emphasis on positions directly contacting the reference position, lower emphasis on positions that indirectly contact the reference position, and minimal or no emphasis on positions far in space from the reference position. In the simplest embodiment, proximity is binary—that is, positions have proximites of 1 or 0, as in Equation 1b.

$$Equation 1 b \text{:}$$

$${proximity}_{ij} = \{ \begin{matrix}1 , if {d}_{ij} \leq cutoff distance \\ 0 , otherwise\end{matrix}$$

In some embodiments of Equation 1b, the cutoff distance may be defined such that only amino acids in direct contact with the reference position have nonzero proximities. To achieve this, the distance cutoff should be in the range of 3-6 angstroms. However, in some embodiments direct contact is best confirmed or established using visual inspection of the structure.

In a preferred embodiment, amino acid similarity is calculated using an amino acid similarity matrix. Such scoring methods, well known in the art of bio-informatics, can be used to quantify the extent of similarity between two amino acids. Similarity matrices, including but not limited to BLOSUM62, provide a quantitative measure of the compatibility between a sequence and a target structure, which can be used to predict non-disruptive substitution mutations (Topham et al., 1997, Prot. Eng. 10: 7-21). Similarity matrices include, but are not limited to, the BLOSUM matrices (Henikoff & Henikoff, 1992, Proc. Nat. Acad. Sci. USA 89: 10917), the PAM matrices, the Dayhoff matrix, and the like. For a review of similarity matrices, see for example Henikoff, 1996, Curr. Opin. Struct. Biol. 6: 353-360. Similarity matrices may also be based on specific properties of amino acids such as hydrophobicity, volume, charge, polarity, polarizability, or isostericity. In some embodiments, amino acid similarity can be replaced with the binary comparison of amino acid identity: nonidentical amino acids have scores of 0 and identical amino acids have scores of 1.

In a preferred embodiment, structural proximity values and amino acid similarity measures are combined to determine the structural environment similarity score (esim) between the template protein and each related protein (in the MSA) at a specified reference position. In a preferred embodiment, this environment score is calculated as in Equation 2, where s is the MSA sequence, i is the reference position, aaⱼ^{s} is the amino acid at position j of sequence s, and aaⱼ^{template} is the amino acid at position j of the template sequence (note that position numbers i and j are defined according to the MSA, not according to the numbering of each individual protein), and the score is a sum over all positions in the sequences.

$$Equation 2 \text{:}$$

$$esim ( s , i ) = {\sum }_{j \neq i}^{positions} {proximity}_{ij} \cdot {S}_{{aa}_{j}^{s} , {aa}_{j}^{template}}$$

where S is a similarity score for comparing the similarity of two amino acids, e.g. BLOSUM62. An optional position-specific weighing function can be added to the above summation to change the influence of different positions in the alignment. For example, the weighing function may be added to change the influence of positions buried in the interior of the protein relative to the surface-exposed residues. The position-specific weighing function can alternatively be used to isolate positions of interest by assigning a zero value to other positions. Positions of interest may include buried positions, exposed positions, positions for which a charged residue is present in the MSA, or positions near a binding site. In preferred embodiments, the structural proximity of adjoining backbone residues is reduced or set to zero (i.e., j≠i is replaced by |j-i|>w in Equation 2, with w ranging from 1 to 10) in order to emphasize three-dimensional structure information over local backbone structural information.

In an alternative embodiment, the environment similarity score (esim) may be calculated as in Equation 2b, where a binary identity comparison is used, in contrast to the use of a similarity matrix in Equation 2.

$$Equation 2 b \text{:}$$

$$esim ( s , i ) = {\sum }_{j \neq i}^{positions} {proximity}_{ij} \cdot {δ}_{{aa}_{j}^{s} , {aa}_{j}^{template}}$$

As will be appreciated, the combination of the simplest form of proximity (Equation 1b) with Equation 2b will result in binary environment similarity scores. That is, an environment is scored to be identical or not.

In additional embodiments, the environment similarity of a MSA sequence can be used as a look-up score for identifying the most similar sequence to the given sequence near the reference position or patch. In this manner, the present invention is useful in a manner similar to BLAST (National Center for Biotechnology Information, National Institute of Health, USA, Altschul, S. F. et al. (1990) J. Mol. Biol. 215:403-410.) or other algorithms that identify similar proteins to a given protein from a large database.

In some embodiments, the environment similarity score can be used directly to generate the final weights of each sequence in the alignment. In preferred embodiment, the final weights are generated by an additional amplification function (such as an exponential), then normalized such that all weights sum to a total probability of 1. In equation 3, an exponential amplification is used with a temperature factor (T) to modulate the extent of amplification, with an optional sequence-dependence weight h(s) for each sequence in the MSA that is used to further bias the influence of some sequences (e.g. as in Henikoff and Henikoff, J Mol Biol. 1994 Nov. 4; 243(4):574-8).

$$Equation 3 \text{:}$$

$$weight ( s , i ) = h ( s ) * \frac{{ⅇ}^{esim ( s , i ) / T}}{{\sum }_{{s}^{′} = 1}^{N} {ⅇ}^{esim ( {s}^{′} , i ) / T}}$$

The sequence-dependent weighing function, h(s), can be used, for example, to change the influence of sequences with a preferred property. These preferred properties include, for example, favorable binding characteristics to another protein, co-factor, substrate, macromolecule, or other entity, favorable in vivo pharmacodynamic properties, favorable activity characteristics, or favorable expression, stability, resistance to aggregation, solubility, or proteases, or structural similarity. Preferred properties need not be limited to physical properties.

Once weights for each sequence at each reference position are calculated, amino acid probabilities can be generated for each reference position with a weighted count over amino acids found at this position in the MSA. These probabilities are referred to as the structure-weighted probabilities or structure-weighted frequencies.

$$Equation 4 \text{:}$$

$$f ( aa , i ) = {\sum }_{s = 1}^{N} weight ( s , i ) \cdot {δ}_{aa , {aa}_{i}^{s}}$$

wherein, δ is the Kronicker delta function, defined such that δ_{xy}=1 if x=y and δ_{xy}=0 if x≠y.

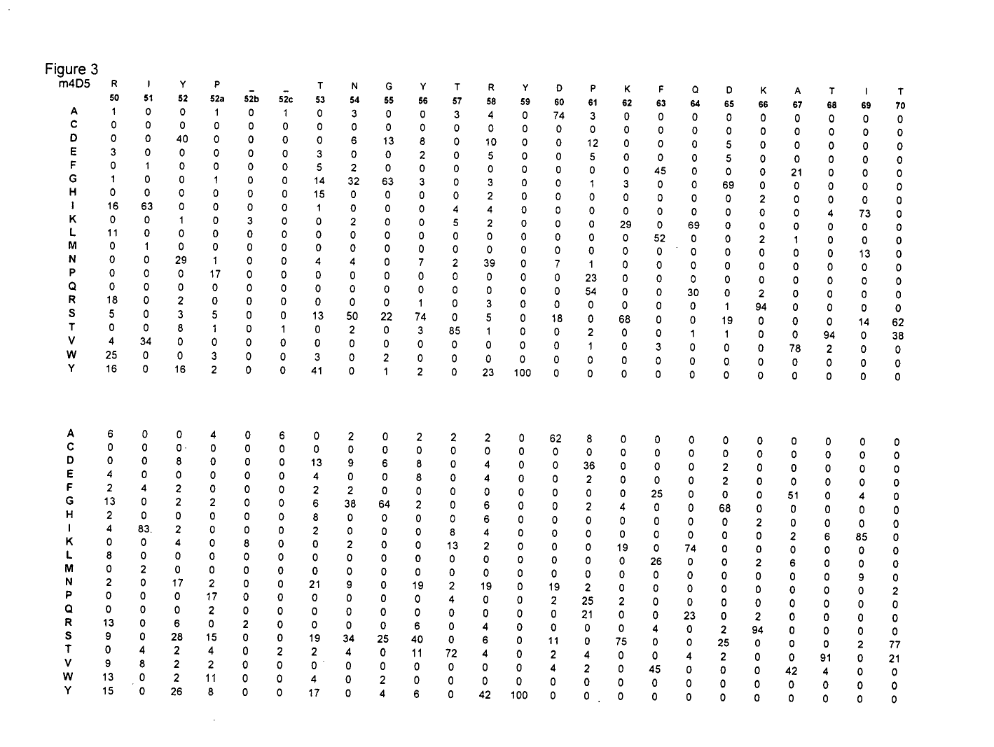

These structure-weighted frequencies differ from the simple amino acid frequencies commonly used. As in known in the art, the amino acid frequency in each position of the MSA can be used to identify a common amino acid for that position. Because the present invention uses structure-weighted frequencies, the output reflects the compatibility of an amino acid in a particular three-dimensional environment. Structure-weighted frequencies at many positions in the antibody heavy chain are compared to typical frequencies in FIG. 3. In that example, the structure-weighted frequencies are shown in the top panel whereas the unweighted frequencies are in the lower panel. The frequencies are shown as percentages for heavy chain positions 50-70.

In an alternative embodiment, the amino acid probabilities f(aa,i) are converted to log-odds ratio scores using Equation 5.

$$Equation 5 \text{:}$$

$$Lscore ( aa , i ) = log ( \frac{f ( aa , i )}{q ( aa )} )$$

where Lscore(aa,i) is log-odds ratio score of amino acid aa at position i based on environment-based sequence weights. The denominator, q(aa), is the overall frequency of amino acid aa found in general. This frequency can be taken from amino acid frequencies calculated from a variety of sources, include the amino acid frequencies found in all proteins in the MSA, all proteins in a particular organism, or all known proteins in every organism, or all proteins in a protein sequence database (e.g. swissprot).

In an alternative embodiment, the relative environment similarity of a sequence at reference position i is calculated relative to a “perfect” environmental match as determined with the template sequence itself, as follows.

Equation 6:
resim(s,i)=e^{(esim(s,i)-esim(template,i))/T}

Where esim(s,i) is determined as in Equation 2.

Thus, a resim(s,i) value of 1.0 implies that, at reference position i, MSA sequence s has a structural environment identical to that of the template sequence. This alternative scoring system is useful for determining structural precedence for each possible amino acid at each position within the template sequence. In a preferred embodiment, the structural precedence for each amino acid at position i is quantified using the highest resim(s,i) value for all aligned sequences {MSA} that possess that amino acid at position i

$$Equation 7 \text{:}$$

$$precedence ( aa , i ) = {Max}_{s ∈ MSA} ( resim ( s , i ) \cdot {δ}_{aa , {aa}_{i}^{s}} )$$

Alternatively, precedence for each amino acid at position i is scored using amino acid similarity instead of identity, as in Equation 7b.

$$Equation 7 b \text{:}$$

$$precedence ( aa , i ) = {Max}_{s ∈ MSA} ( resim ( s , i ) \cdot {S}_{aa , {aa}_{i}^{s}} )$$

Alternative precedence could also be calculated using the weight(s,i) as calculated in equation 3:

$$Equation 7 c \text{:}$$

$$precedence ( aa , i ) = {Max}_{s ∈ MSA} ( weight ( s , i ) \cdot {δ}_{aa , {aa}_{i}^{s}} )$$

As will be appreciated by those in the art, a variety of possibilities exist for scoring precedence, defined to quantify the extent to which a particular amino has already been observed in a protein in the context of a particular structural environment. The functional forms of Equations 2 and 6 also have the advantage that positions with a small number of proximal neighbors (i.e. positions at or near the surface of the protein) will generally have higher precedence values, consistent with expectations of the art.

In additional embodiments, Bayesian statistics methods can be used to further enhance the analysis, particularly when the MSA contains a small number of sequences (e.g., as in Sjolander et al., 1996, Comput Appl. Biosci. 12(4): 327-345). The use of Bayesian statistics allows the introduction of prior information to augment the analysis of amino acid frequencies (as calculated in equation 4). In preferred embodiments, prior information is incorporated using Dirichlet mixtures. In alternate embodiments, prior information may be incorporated using pseudocounts, similarity matrix mixtures, or a common ancestor analysis.

In additional embodiments, structure-weighted frequencies f(aa,i) and precedence(aa,i) or Lscore(aa,i) can be averaged or summed over the whole sequence to generate a composite score that incorporates information from all positions. Methods of averaging, include, but are not limited to, geometric mean, algebraic mean, sum of squares, and other methods.

In preferred embodiments, information from structure-weighted frequencies, precedence scores, environment similarity scores, and averages thereof are utilized to predict or select novel protein sequences with favorable properties. That is, the information can be used to select appropriate modifications to the template protein or any of the related proteins with which it was compared. In a preferred embodiment, amino acids with scores above a user-specified threshold value are selected for substitution into the reference position. This can be done for one or more reference positions. In alternative embodiment, amino acids with the highest scores are selected for substitution into the reference position(s). In additional embodiments, modifications with scores that rank within a given percentile of scores can be used to guide the selection of modifications. In a preferred embodiment, modifications with scores that rank within the top 10% of scores are selected for substitution. In alternative embodiments, modifications with socres that rand within the top 50% of scores are selected for substitution. It will also be appreciated by those in the art that in some cases, where other constraints apply, the user may use output from the method to make a more subjective determination of the most appropriate amino acid substitutions. In less likely but possible embodiments, modifications with particularly low scores can be selected (e.g. if testing hypotheses, or if dramatic perturbation of structural properties is desired).

Consensus Sequence Generation

One embodiment of the present invention is the calculation of a consensus sequence to represent the family of proteins in a MSA. Consensus design is based on the use of a single consensus sequence to represent a MSA. A generic approach to constructing a consensus sequence is to take the most frequently observed amino acid at equivalent positions in the MSA. This is equivalent to constructing the sequence with the maximum probability of being generated using the observed amino acid distributions at each position. That is, of all possible sequences, the consensus sequence is the one that maximizes the quantity, Z, shown in equation 8.

$$Equation 8 \text{:}$$

$$Z = {\sum }_{i}^{positions} log {f}_{MSA} ( aa , i )$$

where aa is a consensus amino acid and f_{MSA}(aa,i) is the frequency of observing aa at position i in the MSA. However, consensus sequences constructed in this manner can contain amino acids in foreign contacting environments since the process of collecting the most frequently observed amino acid at a position does not consider other positions. The present invention adds two important features to this type of analysis. First, the similarity of the consensus sequence to each sequence in the MSA is considered and contributes to the weighted frequency count. Second, and most importantly, three-dimensional structure information contributes to the weighting procedure: similarities between the consensus sequence and each MSA sequence are included with increased weight for positions that are structurally proximal to each reference position. The ACE-based consensus sequence can be constructed by finding the amino acid sequence with the maximum environment-weighted probability, Z^{ace}, as described in equation 9.

$$Equation 9 \text{:}$$

$${Z}^{ace} = {\sum }_{i}^{positions} log {f}_{ACE} ( aa , i \vert consensus )$$

where f_{ACE}(aa,i|consensus) is determined using Equation 4 as discussed earlier. These environment-weighted probabilities depend on the context of the surrounding residues, which necessitates an iterative procedure for determining the ACE-based consensus sequence. In preferred embodiments, a simulated annealing procedure is used to solve for the consensus amino acid sequence that maximizes the quantity found in equation 9. In alternative embodiments, genetic algorithms or Tabu search may be used to solve for the consensus amino acid sequence. In alternative embodiments, steepest-descent or conjugate-gradient minimization techniques can be used from a consensus starting point to generate a modified or corrected consensus.

Patch Mode

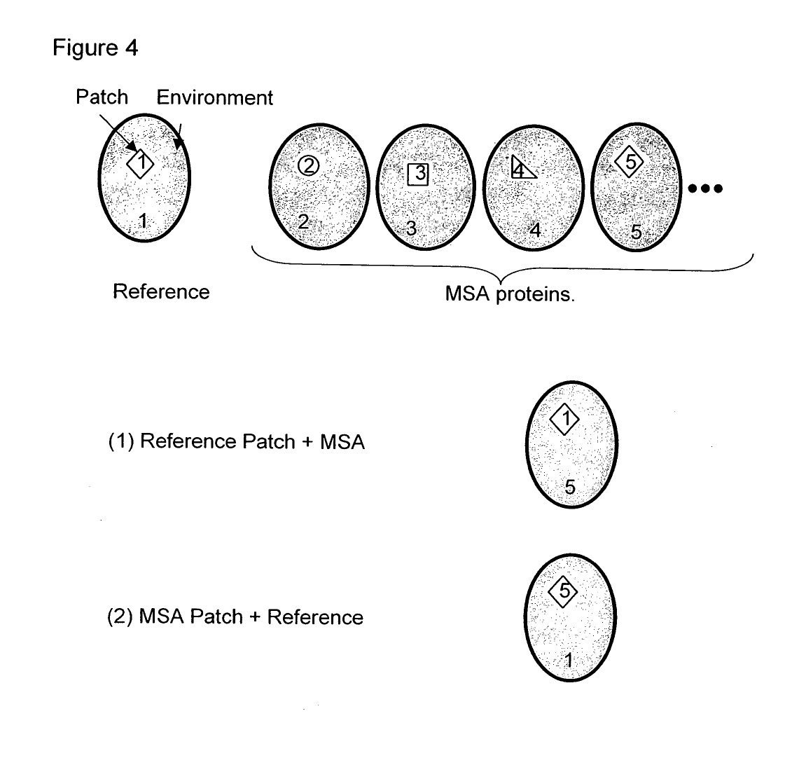

An alternative embodiment of the present invention is the assessment of the compatibility of a group of amino acids, or patch of amino acids, with given structural environment (FIG. 4). This manner of implementing the present invention may be referred to as “Patch Mode”. In a preferred embodiment, the resim score is used to judge the fitness of the patch from one protein and the environment of related proteins, although the structure-weighted frequency and precedence scores can also be used. In this embodiment, the user specifies the patch, a set of positions in the parent protein, and the algorithm calculates a resim score for each sequence in the MSA. The patch may be any number of positions from 1 to the total number of positions in the multiple sequence alignment minus 1 position, with 2-30 residues being commonly used. The resim score reflects the extent of similarity of the environment around the patch in the parent protein structure to the environment found in each related protein in the alignment. It should be emphasized that it is not necessary for the patch residues to be continuous in sequence or nearby in the three-dimensional structure, although the latter is a commonly used.

Information from the patch analysis can be used to selected suitable replacement patches from related proteins that have similar structural environments to the parent protein, and the parent protein can be modified accordingly to generate a second protein. Alternatively, once a related protein with a similar patch structural environment is discovered, it can be selected as a host in which to graft the parent protein patch. The choice of direction depends on the intended effect of the modification.

The use of many residues in the patch requires only slight adjustments to the equations described above wherein the “patch” was only one position, the reference position i. With multiple residues in the patch, the environmental similarity score is calculated similarly (equation 10).

$$Equation 10 \text{:}$$

$$esim ( s , P ) = {\sum }_{j ∉ P}^{positions} {proximity}_{Pj} \cdot {S}_{{aa}_{j}^{x} , {aa}_{j}^{template}}$$

“s” still refers to the MSA sequence in question, and “S” still refers to the similarity of each non-patch amino acid in the template and sequence “s” at position “j”. In Patch mode, however, the “P” now refers to a set of patch residues. The summation is done over all positions, j, that are not included in the patch set of residues.

Previously, the proximity(i,j) value was the proximity of residue j and the reference amino acid, i. In patch mode, the proximity (P,j) is commonly taken as the largest proximity value found between residue j and any residue in the patch. That is:

$$Equation 11 \text{:}$$

$${proximity}_{Pj} = {max}_{k ∈ P} ( {proximity}_{k , j} )$$

Other methods of determining the proximity of the patch and an environment residue are also suitable. For example, the average proximity, or minimum proximity, of the patch residues to the environment residues may be used as may the sum of the proximities from each patch residue to the environment residue.

An additional embodiment of the present invention is the use of patch mode to calculate structure-weighted frequencies for a patch in a manner analogous to the calculation of structure-weighted frequencies for individual residues. In patch mode, the structure-weighted frequencies are the frequencies of finding a certain set of amino acids in the given patch positions. For example, if one is interested in placing both an Ala and an Arg at two positions (the patch) of a protein, higher structure-weighted frequencies found for the Ala and Arg pair than for another pair of amino acids would indicate that the Ala and Arg pair is a more favorable substitution. Equations 3 and 4 are used as before, being slightly modified to reflect the use of the patch of amino acids, as shown in equations 12 and 13.

$$Equation 12 \text{:}$$

$$weight ( s , P ) = h ( s ) * \frac{{ⅇ}^{esim ( s , P ) / T}}{{\sum }_{{s}^{′} = 1}^{N} {ⅇ}^{esim ( {s}^{′} , P ) / T}}$$

$$Equation 13 \text{:}$$

$$f ( paa , P ) = {\sum }_{s = 1}^{N} weight ( s , P ) \cdot {δ}_{paa , {paa}_{P}^{s}}$$

where weight (s, P) is the weight of MSA sequence s given reference patch P, paa is the particular patch of amino acids for which a frequency is being determined, paa^{s}ₚ is the patch of amino acids found in MSA sequence s at the positions found in patch P, and f(paa,P) is the structure-weighted patch frequency. As written above, the Kroniker delta function would equal to 1 if all the amino acids in the patch residues of sequence s match all the amino acid for which the frequency is determined. That is, for example, if the user is interest in placing Ala and Arg into two patch positions simultaneously, the sequence weights are added over all sequences in the MSA that have the Ala and Arg at the two patch positions.

The requirement that all amino acid in the sequence s patch are equivalent to all the amino acids for which the frequency is being determined is often overly restrictive. This requirement can be made less restrictive by substituting other functions for the Kroniker delta function. Useful substitute equations include ones that determine the percent identity or percent homology of the two patches, in which similarity matrices like BLOSUM or PAM matrices may again be used.

Precedence scores may also be used with a patch of residues designated. In this case, the precedence score demonstrates that at least one MSA sequence has an environment surrounding the patch that is similar to the environment found in the template sequence. The precedence score is determined in a similar manner to the instances in which only one reference residue exists, ie the patch has only one residue.

$$Equation 14 \text{:}$$

$$precedence ( aa , P ) = {Max}_{s ∈ MSA} ( resim ( s , P ) \cdot {δ}_{aa , {aa}_{t}^{s}} )$$

An additional embodiment of the present invention is the calculation of the similarity of the amino acids in the parent patch residues to the amino acids in the patch residues of each related sequence. In prior embodiments, the similarity of the environment in the template sequence to the environment in each MSA sequence was used to judge the fitness of the patch and the environment created by each MSA sequence. Particularly with patches consisting of larger numbers of positions, the similarity of the amino acids in the template patch and each MSA patch can be used to further judge the suitability of a patch and an environment. The similarity of patch residues in the template sequence and each MSA sequence can be calculated by various methods. One simple method is simply to sum the similarity scores of the template and MSA sequence amino acids over every position found in the patch, namely

$$Equation 15 \text{:}$$

$$patchsim ( s ) = {\sum }_{p ∈ patch} {S}_{{aa}_{p}^{s} , {aa}_{p}^{template}}$$

wherein, s refers to a sequence in the MSA, S refers to the similarity of two amino acids, p is a position in the patch. Alternatively, the analog of Equation 6 can be used to provide a relative patch similarity measure. These measures of patch similarity can be augmented with terms, for example, that take into consideration the proximity of the patch residue to the environment residues, the overall similarity of the MSA sequences and position- and sequence-specific weighing functions as was done in the comparison of environmental residues described herein. More additions can be incorporated, making the patchsim score more and more similar to the previous environment similarity score.

In short, the designation of which positions are “patch” residues and which are “environment” residues is left to the user. By convention, we typically describe the algorithm as calculating the similarity of the environment residues in the template and a MSA sequence. The current invention may be used twice to gain more information useful in protein design. A patch may be given as a set of resides and the current invention used to compare the similarity of the environmental positions around the patch. Then, the current invention could be used again with the patch residues being defined as those residues in the environment of the preceding use.

Optimization of ACE™ Technology

In a preferred embodiment, optimal equations and/or parameters for distance-to-proximity conversion, temperature factor, environment similarity, etc. can be selected by systematic evaluation of the effect of equation/parameter choice on the predictive performance of the method. In a preferred embodiment, the present invention is optimized so that results are in accordance with existing experimental mutational data sets. The parameters of the present invention may include, but are not limited to, the form of the proximity function (equation 1), the proximity scale factor σ (equation 1), the temperature scale T for esim (equations 3 and 6), and the selection of the similarity matrix S (equation 2). In a preferred method, parameters are chosen to maximize the correspondence of ACE™ amino acid probabilities, log-odds ratio scores, or precedence scores with experimentally determined sequence descriptors that may include stabilities, binding affinities, expression levels, other descriptors, or combinations of sequence descriptors. The correspondence may be measured in various manners including a correlation coefficient, the area under a receiver-operator curve, a P-value, or a Matthew's correlation coefficient.

Virtual MSAs

An additional embodiment of the current invention is the analysis of a virtual MSA generated by automated computational protein design algorithms, such as Protein Design Automation (PDA®, patent reference here) and Sequence Prediction Algorithm (SPA™, patent reference here) technologies. The application of virtual MSAs is especially important for proteins that have very few natural homologues with which to create a MSA. Computationally generated MSA's may also be generated with unnatural amino acids. In this case, the ACE™ technology may be used to build up, from the virtual MSA, a diverse set of sequences similar to the protein of interest for the given reference position or patch.

### EXAMPLE 1

Human Heavy Chain Sequences

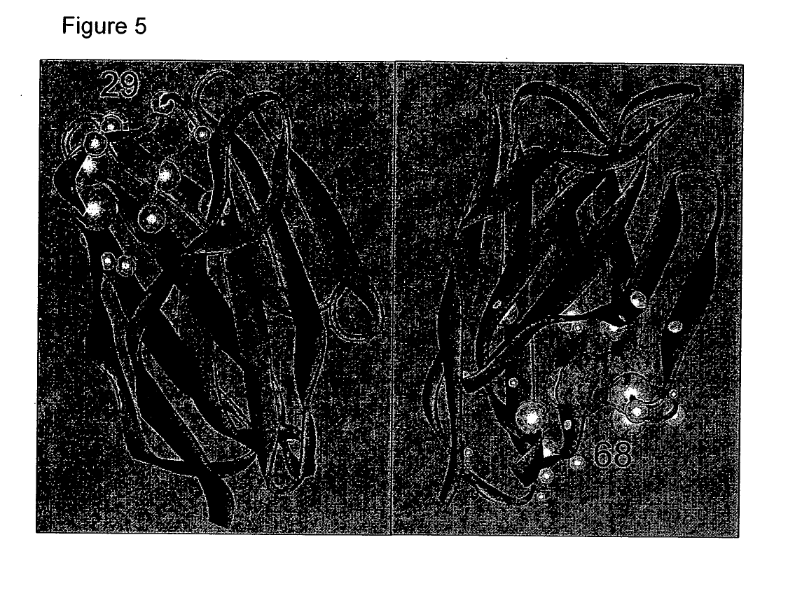

The antibody heavy chain sequences can be aligned and used with an existing structure as input into the present invention. FIG. 5 shows the structure of Herceptin® (trastuzumab) (Genentech/Biogenldec) (PDB code 1FVC) and proximity values determined by an embodiment of the present invention. The left panel shows proximities values determined when position 29 is designated as the reference position or patch. The amino acid of position 29 in the reference structure is shown as a non-spherical surface. The remaining positions in the protein, the environment positions, are shown as a spheres positioned on their C positions in the structure. The volumes of the spheres are proportional to their proximities to position 29. Larger sphere indicate more proximal environment positions, which are weighted more strongly in the determination of the structure-weighted frequency and precedence scores. The right panel shows the proximity values determined when position 68 is the patch, or reference, position.

### EXAMPLE 2

Sequence Weight Determination

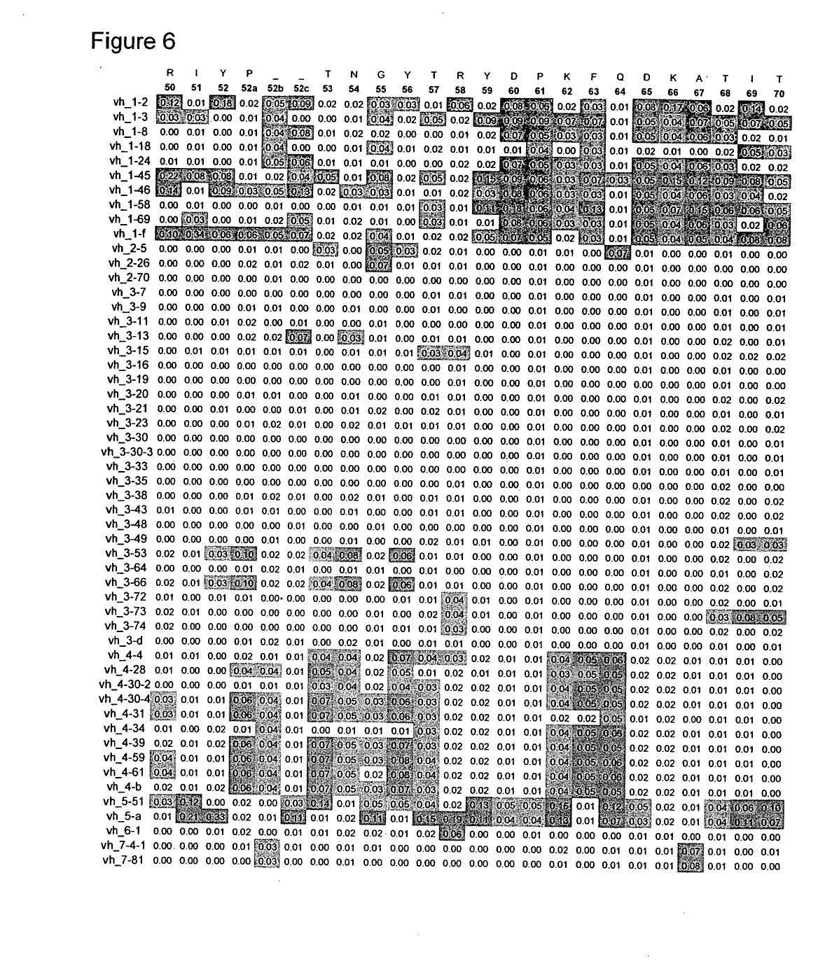

An alignment of human heavy chain germline sequences, the reference sequence, m4D5, and the structure, PDB code 1FVC, was used to determine the sequence with the most suitable environment around each position in the multiple sequence alignment (MSA). FIG. 6 shows the sequence weights calculated with equation 3 using a temperature (T) value of 1, the BLOSUM62 (Henikoff J. G. Proc. Nat Acad. Sci USA 89:10915-10919 (1992)) similarity matrix (eq. 2) and a σ value of 5 (eq 1). The figure illustrates how the similarity of each sequence to the reference sequence depends upon the position given as a reference position. For example, the environment around position 50 in sequence vh_{—}1-45 is the most similar (similarity score=0.22) to the reference environment of all the listed sequences.

### EXAMPLE 3

Patch Mode—Multiple Residues Considered.

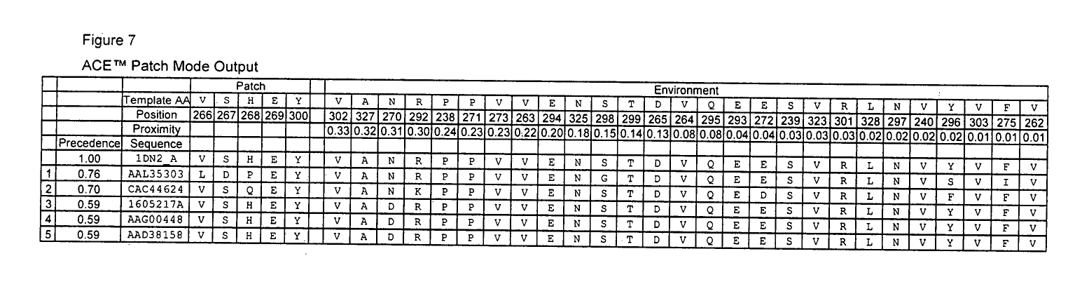

ACE™ algorithms are useful in patch mode to determine the best environment in which to place a patch of amino acids, or to determine the best patch of amino acids to place into a particular environment. A template structure and a multiple sequence alignment comprising the sequence of the template structure are input as are a list of residue positions defining the patch. FIG. 7 shows the ACE™ resim scores determined using a multiple sequence alignment of antibody Fc domains and an Fc structure, PDB code 1 DN2. The multiple sequence alignment used was generated with BLAST (Altschul, S. F., et al. (1990) J. Mol. Biol. 215:403-410.) using the sequence of the human IgG1 Fc domain as input. The multiple sequence alignment contained 249 positions (residue plus gaps) and 137 sequences including the sequence of the template structure. Henikoff weights (Henikoff & Henikoff, 1992, Proc. Nat. Acad. Sci. USA 89: 10917) were applied to the sequences to reduce the influence of very similar sequences. User-defined position specific weights were not used, allowing the proximity values to determine the contribution of each environment residue to the environment.

For this example, a patch was chosen using residues 266, 267, 268, 269 and 300. The 27 environment residues closest to the patch residues are shown with their proximity values on the right side of the figure. V302 is the closest environmental residue to the patch having a proximity value of 0.33. The top 5 sequences with the best environment for the patch are shown under the sequence of the template structure. These sequences gave a high precedence score. The top ranking sequence, labeled “AAL35303”, has an environment that differs from the environment from the template sequence in that it contains a Gly at position 298 in place of a Ser. This change, and the other less proximal changes, drops the precedence score below the value of 1.0, which is found only in an exact match.

### EXAMPLE 4

CDR Grafting.

One example of the use of the current invention is found in CDR grafting. In CDR grafting, the complement-determining regions (CDRs) of the variable region of an antibody, say a murine antibody, are substituted into another antibody, say a human antibody. This procedure produces an antibody that possesses the antigen-binding specificity of the murine antibody and has human-derived sequences in the remaining positions to reduce the stimulation of an immune response in human patients. In the case of the antibody heavy chain, the researcher must decide which of the many possible human heavy chain sequences would be the best choice to accept the graft of the murine CDRs. Choosing a compatible human heavy chain acceptor will minimize the losses in antigen binding affinity, which frequently accompany CDR grafting.

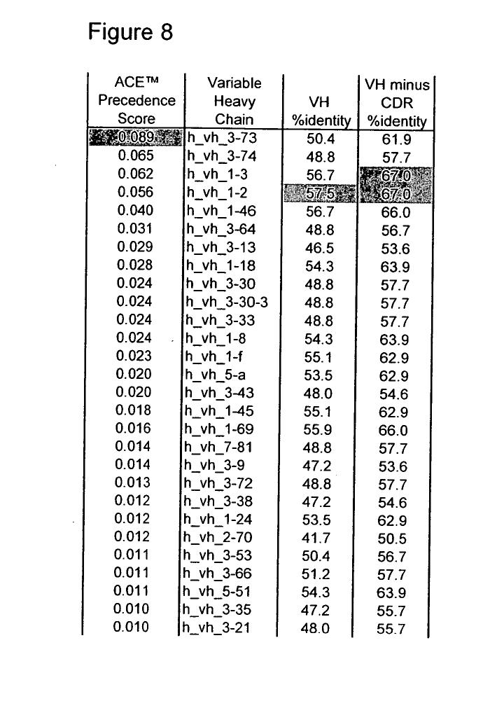

FIG. 8 shows the compatibility of the CDRs of a murine heavy chain antibody used by Carter et al. (1992 Proc Nat. Acad. Sci. USA 89(10):4285-9) with many possible acceptor human antibody germline sequences. Surprisingly, the methods of the present invention demonstrate that the most compatible human sequence is that of h_vh_{—}3-73, whereas other human sequences, namely h_vh_{—}1-2 and h_vh_{—}1-3, would be chosen base on the percent sequence identity. The percent sequence identity measure identifies the human sequence that is most similar to the murine sequence overall. The methods of the present invention, however, include the structure in the analysis and identify the human sequence that is most similar to the murine sequence in the regions proximal to the CDRs.

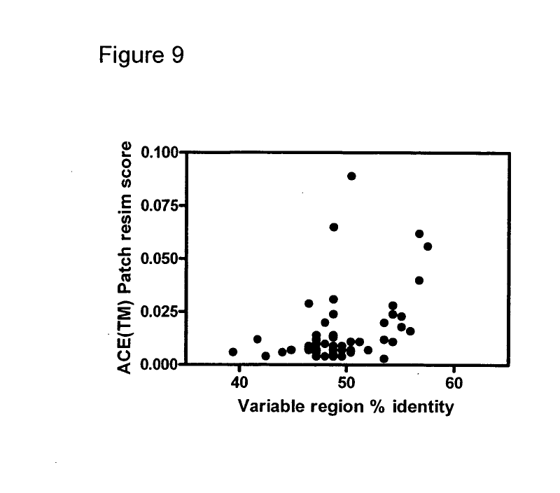

FIG. 9 shows the poor correlation of the resim scores and the overall percent identities of the human sequences to the murine sequence. (In this graph the percent sequence identity was calculate using all residues in the variable heavy domain. Using only those residues not found in the CDRs produces a similar graph, as predicted from the strong correlation of columns 3 and 4 in FIG. 8.) The two human sequences with the highest resim scores, h_vh_{—}3-73 and h_vh_{—}3-74, have percent identities that are not significantly above the average percent identity for the 52 human sequences. Therefore the methods of the present invention suggest a human acceptor sequence that is not the optimum sequence as judge by the similarity of the murine sequence to the human sequences overall.

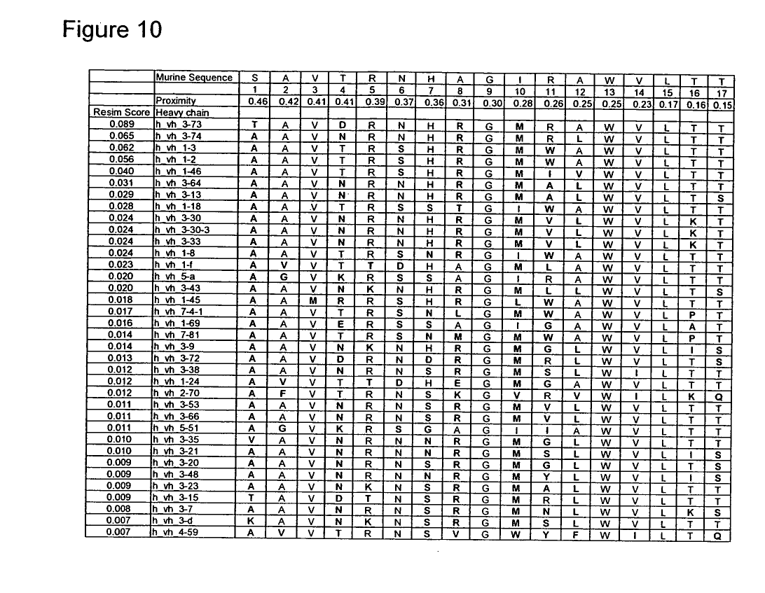

By looking at the environment residues most proximal to the CDRs, we may identify the residues in h_vh_{—}3-73 that gives it a favorable resim score for accepting the murine CDRs. FIG. 10 shows the human heavy chain sequences and their resim scores. One column of the table shows the amino acids found in one environment position and the position's proximity to the CDR patch. The closest environment residue, which is Ser in the murine sequence, has a proximity of 0.46 from the patch. The most favorable heavy chain sequence, h_vh_{—}3-73, has a Thr at this position whereas most other heavy chain sequences have a Ala at this position. h_vh_{—}3-73 has a more favorable resim score than say, h_vh_{—}3-74, because Thr is a more conservative substitution for Ser than is Ala. Amino acid differences at other positions also influence the resim scores, but those differences are weighted less because of their lower proximity.

Whereas particular embodiments of the invention have been described above for purposes of illustration, it will be appreciated by those skilled in the art that numerous variations of the details may be made without departing from the invention as described in the appended claims. All references cited herein, including patents, patent applications (provisional, utility and PCT), and publications are incorporated by reference in their entirety.

1. A method of grafting a complementarity-determining region (CDR) of a donor variable domain into an acceptor variable domain comprising:
a) providing an amino acid sequence of a donor variable domain, the donor variable domain comprising a donor CDR and donor non-CDR amino acids;
b) providing a plurality of amino acid sequences of acceptor variable domains, the acceptor variable domains each comprising an acceptor CDR and acceptor non-CDR amino acids;
c) providing structural data comprising a reference set of a plurality of distances, each distance being measured between a first amino acid and a second amino acid of the donor variable domain;
d) calculating (i) a distance-weighted similarity score for the donor variable domain and (ii) a plurality of distance-weighted similarity scores for the acceptor variable domains;
e) calculating a plurality of suitability scores for the acceptor variable domains using the similarity scores;
f) creating a ranking of the plurality of suitability scores;
g) choosing a compatible acceptor variable domain based on the ranking; and
h) replacing the CDR of the compatible acceptor variable domain with the CDR of the donor variable domain.

2. The method of claim 1, wherein said distance-weighted similarity scores are calculated such that residues within about 10 Angstroms of the residues of the CDRs are given full weight and residues not within about 10 angstroms are given zero weight.

3. The method of claim 1 or 2 wherein said donor domain and said acceptor domains comprise heavy chain variable domains.

4. The method of claim 1, 2 or 3 wherein said donor domain and said acceptor domains comprise light chain variable domains.

5. The method of claims 1-4 wherein the variable domain is contained within an antibody.

6. The method of claims 1-5 wherein said compatible acceptor variable domain is from a human antibody.

7. The method of claims 1-6 wherein said CDR of said donor variable domain is murine.

8. The method of claims 1-7 wherein said acceptor variable domains are human germline variable domains.

9. The method of claims 1-8 wherein said distance-weighted similarity score utilizes weights calculated as a non-discrete function of distance.

10. The method of claims 1-8 wherein said distance-weighted similarity scores are calculated wherein the weights of residues are inversely proportional to the distance of said residues from the residues of the CDRs.

11. The method of claim 10, wherein said weights of residues decrease exponentially with the distances of residues to the CDRs.

12. The method of claim 10, wherein said weights of residues decrease linearly with the distances of residues to the CDRs.

13. The method of claim 10, wherein said distance-weighted similarity scores are resim scores.
